(19) United States
(12) Patent Application Publication
(10) Pub. No.: US 2021/0228390 A1
(43) Pub. Date: Jul. 29, 2021

(54) One-arm implement for user with disabled arm.

(71) Applicant: Leslie G. Shepherd, Ewing, NJ (US)

(72) Inventor: Leslie G. SHEPHERD, Ewing, NJ (US)

(73) Assignee: Leslie G. Shepherd, Ewing, NJ (US)

(21) Appl. No.: 16/873,066

(22) Filed: Jan. 27, 2020

(51) Int. Cl. A61F 4/00

(52) U.S. Cl. CPC A61F 4/00

(57) ABSTRACT

A one-arm implement for a user with a disabled arm includes: a) a work function implement having an elongated pole with an upper section and a lower section, including a handle portion, and having a work function tool located at its distal end; b) a forearm receptacle located on the upper section of the pole; c) a hand grip located on the upper section of the pole and positioned downwardly from the forearm receptacle; d) at least one foot push plate located on at least one of: the lower section of the pole; and the work function tool.

### REFERENCE TO RELATED APPLICATIONS

The present application is not related to any pending or issued United States of America or foreign patent or patent application.

### BACKGROUND OF INVENTION

### a. Field of Invention

The present invention generally relates to products developed to assist permanently and temporarily handicapped people having only one functional arm, to perform labor functions of pushing and/or lifting that would normally require two arms. The arm assist products are, in some embodiments, retrofit devices that have three critical features- a forearm receiver, a hand grip, and an attachment feature. These retrofit devices may be attached to any long-handled tool, such as a snow shovel, a digging shovel, a broom, a mop, a brush, a paint roller, etc. In yet other embodiments, the present invention is any of the foregoing or similar tools with the aforementioned arm assist aspects built into or onto the products when they are manufactured. These latter present invention products may preferably include a foot pedal to assist in pushing.

### b. Description of Related Art

The following patents are representative of the field pertaining to the present invention:

U.S. Pat. No. 9,573,265 B2 to Li relates to an arm holder for a pole-handled tool such as a broom, a mop, a squeegee, etc. that allows effective use of the pole-handled tool with only one hand. The arm holder comprises an arm grip having an arm-holding structure; and an attachment portion capable of attaching the arm grip to the pole-handled tool; wherein the arm-holding structure is capable of receiving an arm positioned within the arm-holding structure while a corresponding hand of the same arm grasps the pole handle, the pole handle acting as a lever where the corresponding hand forms a fulcrum of the lever.

U.S. Pat. No. 9,635,796 B1 to Amirault describes a shovel assembly for coupling a shovel to a forearm that includes a shovel that may be coupled to a forearm of a user. Thus, the shovel may be manipulated to shovel material. A support is coupled to the shovel. The support may to be secured to the user's forearm. Thus, the shovel is retained on the user's forearm.

U.S. Pat. No. 8,464,399 B2 to Herigstad describes an ergonomic anatomical assist system for mounting on a handled receptacle device, having a hand grip disposed on a receptacle end of the device, the hand grip disposed on the device transversely to the handle and extending off the handle so that a user's palm can grasp the grip with palm facing up and opposite the receptacle. The system may further have a forearm support configured to cradle around at least a portion of the user's forearm, the support being disposed on a handle portion of the device that is spaced so as to distribute the load of the device over at least two spaced apart anatomical points, one being at the hand and the other along the forearm.

U.S. Pat. No. 6,464,272 to Michaud describes a design to be used by handicapped persons for the removal of snow. The invention is made to be used with one arm, and is adjustable to fit a short or tall person with only one arm or leg, left or right. It consists of a grip handle which is secured to two plates by bolts which are attached in turn to the main shaft by bolts, and is made adjustable by holes drilled into the main shaft. It also has a cradle at the upper end of the main shaft in which the forearm rests to give it stability and leverage to lift the loaded shovel for throwing snow. This cradle is attached to the main shaft by clamps which are also adjustable by releasing the bolts and moving it up and down on the shaft, which is also used to allow for use of different sized persons in unison with the adjustment of the grip handle.

U.S. Pat. No. 6,082,795 to Fornelli describes an inflatable vehicle occupant protection device module for a vehicle includes an inflatable vehicle occupant protection device having a first panel portion which inflates in a first direction, a second panel portion which, when the vehicle occupant protection device is inflated, is deployed in a second direction transverse to the first direction. A cover for the vehicle occupant protection device has a tear seam which tears during inflation of the inflatable vehicle occupant protection device. The tear seam defines a first door in the cover. The first door swings open in the second direction during inflation of the inflatable vehicle occupant protection device. A connection connects the second panel portion to the first door to cause the first door to pull the second panel portion in the second direction as the first door swings open.

U.S. Pat. No. 5,937,627 to McKittrick describes a hand held garden tool configured to enhance downward force for digging and raking. The tool has a rod frame which extends from a working element attached to the tool at the front end of the tool to a brace located at the rear of the tool. The brace rests on the user's wrist or forearm, so that additional force is developed when the working element is urged downwardly into the ground. A handle depends from the frame at a point between the working element and the brace. Vertical orientation of the handle is more natural and comfortable than would be a horizontal orientation. The handle is provided with a molded grip for cooperating with a user's hand. The tool has a chuck for accepting diverse working heads, and preferably is provided with a plurality of diverse working heads. Exemplary working heads include rake, spade, and pry.

U.S. Pat. No. 5,832,563 to Simpson describes the present invention is directed to a fore-arm assistant device. The device has a frame which encircles at least part of a forearm of a user. A cuff is positioned on one end of the frame so as to encircle the forearm adjacent to the elbow of the user. A handgrip extends from one side of the frame to the other side of the frame and is positioned approximately the length of the forearm from the elbow end of the frame. A tubular locking member is positioned on the other end of the frame. Various tools, a crutch leg or a kayak paddle can be held in the device.

U.S. Pat. No. 5,716,087 to Backich et al describes an ergonomic scoop assembly including a scoop subassembly and scoop support subassembly. The scoop assembly is preferably detachably connectable to the scoop support subassembly. The scoop support subassembly includes a saddle member for contacting the ventral side of a user's forearm and structure for releasably securing the scoop support subassembly to the user's forearm. The scoop support subassembly further includes a rear portion to which the saddle member is attached and a fore portion attached to a handle of the scoop subassembly. When the scoop support subassembly is attached to the user's forearm the handle of the scoop subassembly defines an angle with respect to the longitudinal axis for the user's forearm of between about 115.degree. to about 125.degree. The scoop assembly enables the user to comfortably and effectively perform scooping operations for prolonged periods of time with minimal fatigue and at considerably reduced potential for cumulative trauma injury to the user's upper extremities versus conventional scoop designs.

U.S. Pat. No. 5,592,357 to Hoffman describes a full motion leverage enhancing assembly is provided for attachment to the handle of a mop, broom, rake or similar tool which has an elongate handle and a center of gravity strongly skewed away from the grip point utilized by the tool operator. In this embodiment, the leverage enhancing assembly comprises an arm clamp for securing the tool handle to a single arm of the user and a grasping shaft for grasping the tool handle. The arm clamp is comprised of a first rigid longitudinal segment adjustably attached to the end of the tool handle proximate to the user by a semi-rigid clamp having an adjustable circumference. The arm clamp is further provided with a pair of flexible, adjustably interlocking straps extending from the proximal end of the longitudinal segment opposite the clamp. The straps are constituted to form a semi-permanently and adjustably substantially circular clasp for receiving and securing a portion of the user's forearm. The grasping shaft comprises a second rigid longitudinal segment attached to the tool handle by a semi-rigid clamp opposite the user relative to the arm clamp and in axial alignment with the arm clamp. This second longitudinal segment is furthered configured to be adjustably matable to the first longitudinal segment. The grasping shaft is substantially perpendicularly protruding from the second longitudinal segment and has an abutment flange for increasing the stability of the user's grip on the grasping shaft.

U.S. Pat. No. 5,471,698 to Francis et al describes a hand tool having a frame which holds interchangeable working heads for scraping ice and other materials, scrubbing and sanding. The frame pivotally carries the working head at one end and has a curved forearm brace at the other end. A hand grip bar is located between the ends of the frame such that the forearm provides force against the brace to hold the working head against the work surface. This creates a leverage effect and relieves stress on the wrist. The pivotal head allows angled or contoured surfaces to be followed without requiring the wrist to flex at different angles. The scraping head has two scraping edges which scrape on both push and pull strokes. An accessory head which is interchangeable with the ice scraping head can hold two metal scraping blades or interchangeable base plates which provide a sanding sheet and a scrubbing pad, respectively.

U.S. Pat. No. 5,451,085 to Wagner describes a tool-handle holder has an arm loop (2) that fits on a person's forearm at an arm end of an attachment section (1) and a handle (3) at a hand end of the attachment section (1). The attachment section (1) between the arm loop (2) and the handle (3) is constructed for linear and lateral support of a clamp (4) in combination with a handle (3). The handle (3) is extended from the hand end of the attachment section (1) to a position in a plane that is parallel to the attachment section and bisects the arm loop (2). Basic construction can be achieved from one piece of PVC pipe having a desired diameter and heat-bent to form the handle (3) at the hand end and the arm loop (2) at the arm end of two juxtaposed sections of the PVC pipe which form the attachment section. Various forms of clamps can be attached to contain a handle of a tool or instrument separately from or buttressed against the attachment section. Reinforcement can be placed in the PVC pipe. The attachment section also can be an attachment beam extended between the arm loop (2) and the handle (3). Nonslip means and various forms of clamps can be used with either construction.

U.S. Pat. No. 2,482,589 to Maguire describes a device, in combination, having a body member, handle means connected to said body member adjacent one end thereof, ring shaped means connected to said body member adjacent the other end thereof, said ring shaped means being adapted to fit around the forearm of the user, and a pair of clamps connected to said body member, said clamps being spaced from each other a substantial distance and being positioned on the opposite side of said body member from said handle means and said ring shaped means, said clamps being operable to detachably connect an implement to said body member.

U.S. Pat. No. 712,843 to Paul describes a pneumatic sugar cane cutter with a handle that includes a forward handgrip and a forearm plate with a strap.

U.S. Pat. No. 309,437 to Calef describes a mixing spoon having a shank formed at its upper end with an elastic arm piece and a handle.

United States Patent Application Publication No. 2017/0016194 A1 to Quinn et al describes A snow flipper for clearing snow. The snow flipper includes a blade. The snow flipper also includes a handle attached the blade. The handle includes a first portion, a second portion attached to the second portion and a bend creating a vertical portion of the handle. The snow flipper further includes a handgrip. The handgrip is movably secured to the handle, telescopes relative to the handle and provides a gripping surface for the user. The snow flipper additionally includes a flat spring attached to the vertical portion of the handle. The flat spring includes a curved piece of rigid material configured to deform under a force, return to its initial shape upon removal of the force and cause downward motion of the handgrip to result in motion that is upward and away from the user. The snow flipper moreover includes a wheel attached near one end of the flat spring.

U.S. Design Pat. No. D825,297 to Ahearn describes a gardening tool with a grip and a forearm cup and U.S. Design Pat. No. D807,716 also to Ahearn illustrates a similar garden tool with a grip and a forearm cup.

Notwithstanding the prior art, the present invention is neither taught nor rendered obvious thereby.

### SUMMARY OF INVENTION

The present invention is directed to a product that is a one-arm implement for a user with a disabled arm. By “one-arm implement” is meant an implement that is used with one arm and hand, rather than with two arms and hands. Thus, the present invention includes: an implement for performing a work function, such as shoveling, raking, mopping, sweeping, roller painting, etc. and, a forearm receptacle, a hand grip and at least one foot push plate. In further detail, the present invention includes a) an implement for performing a work function, said implement having an elongated pole with an upper section and a lower section, said upper section including a handle portion, and said lower section having a distal end, and having a work function tool located at said distal end; determining the angle of the meridian axis of astigmatism of said eye relative to a reference axis; b) a forearm receptacle located on said upper section of said pole; c) a hand grip located on said upper section of said pole and positioned downwardly from said forearm receptacle; d) at least one foot push plate located on at least one of: said lower section of said pole; and said work function tool.

In some preferred embodiments of the one-arm implement for a user with a disabled arm, the at least one foot push plate is located on said lower section of said pole. In other preferred embodiments, the at least one foot push plate is located on said work function tool. In others, the said at least one foot push plate is located on both said lower section of said pole and said work function tool. In some of these preferred embodiments, the at least one foot push plate is at least partially located rearward of said on said lower section of said work function tool and forms an angle no greater than 60 degrees when measured at the at least partially located rearward section and up. In others, the at least one foot push plate is at least partially located forward of said on said lower section of said work function tool and forms an angle no greater than 60 degrees when measured at the at least partially located rearward section and up.

In some preferred embodiments of the one-arm implement for a user with a disabled arm, the work function tool is selected from the group consisting of a snow shovel, a digging shovel, a rake, a fork, a broom and a mop.

In some preferred embodiments, the forearm receptacle located on said upper section of said pole, includes at least one opening-adjustment mechanism adapted to provide a user with an adjustable fitting to accommodate different thickness forearms. In these embodiments with the opening-adjustment mechanism, any one or a combination of the features and components described in the previous paragraphs may be included as more preferred embodiments.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE SEVERAL VIEWS OF THE DRAWINGS(S)

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1A is a schematic diagram of the various components, options and features of the present invention one arm implement for a user with a disabled arm;

FIG. 1B is a schematic diagram of details of the forearm receptacle component of the present invention one arm implement for a user with a disabled arm;

FIG. 2 shows a partial side view of an embodiment of a present invention one arm implement for a user with a disabled arm, illustrating the pole, a handle, grip and arm receptacle;

FIG. 3 shows a partial side view of another embodiment of a present invention one arm implement for a user with a disabled arm, illustrating the pole, an upper handle, a grip and arm receptacle;

FIG. 4 shows a front view of one embodiment of a present invention forearm receptacle component having extended internal padding and an adjustment mechanism for inside cross-section adjustment to accommodate different size forearms;

FIG. 5A shows a partial cut side view of a present invention one arm implement for a user with a disabled arm, specifically showing the foot push plate in conjunction with a snow shovel, and FIG. 5B shows a back view thereof;

FIG. 6 shows a side view of and amputee using a present invention one arm implement pitch fork;

FIGS. 7 and 8 show a front, open and closed views, respectively, of another embodiment of a present invention forearm receptacle component having extended internal padding and an adjustment mechanism for inside cross-section adjustment to accommodate different size forearms;

FIG. 9 shows show a front view of yet another embodiment of a present invention forearm receptacle component having extended internal padding and an adjustment mechanism for inside cross-section adjustment to accommodate different size forearms which includes a Velcro type adjustment along with a mouth pull to enable a one arm user to make adjustments with his teeth by lifting and pulling and applying the attachment.

### DETAILED DESCRIPTION OF THE EMBODIMENTS

The present invention one arm implement for a user with a disabled arm may be constructed on any useful materials, although plastics, metals, foams and wood structures are very effective. Generally, a combination of these materials may be used. The pole may preferably be metal tubing, wood poles or plastic extruded or molded poles. The handle may be of the same or different materials from the pole, and the handle may be either an area on the pole, a protrusion or extension of the pole or combinations thereof. The handle enables a two-armed user to use the implement in a conventional manner. The grip is separate from the handle, although it is a species of handle attached to the pole, but is meant to be used by the one arm user in conjunction with the forearm receptacle. The forearm receptacle is preferably adjustable and is ideally made of strong plastic, molded rigid foam, or lightweight metal or a combination thereof, and is preferably padded with a soft foam. The grip and forearm receptacle may be a single component or two separate components, and may be permanently attached to the pole or removably attached (and hence could be a retrofitted product). Thus, the grip and forearm receptacle could be sold for retrofit along with a foot push plate.

The foot push plate may be made of strong plastic, rigid foam or metal and may have sidewalls to inhibit slipping. The foot push plate is attached to either the pole or the working tool implement, or both. The distal end tool implement is a functional tool formed as any present-day tools would be, such as metal and plastic shovels and rakes, metal and plastic paint rollers, natural and synthetic sponges, metal pitch forks, strand and sponge and rubber mops and squeegees, etc.

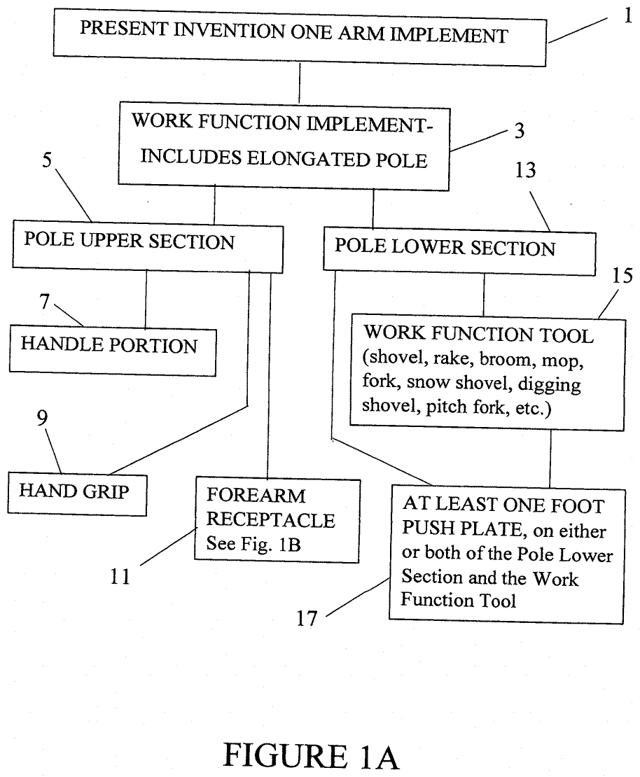

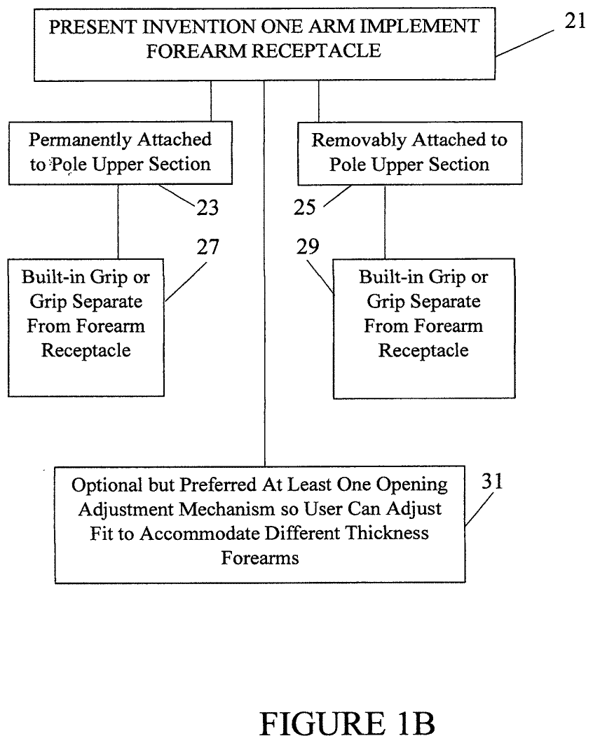

FIG. 1A is a schematic diagram of various components, options and features of the present invention one arm implement for a user with a disabled arm, block 1. It includes a work function implement that includes an elongated pole, block 3. The pole should be interpreted broadly, as it could have any cross-sectional shape and be made of any rigid material(s). It could include a small motor or not, such as a weed whacker or a leaf blower, each with motors, or a mop, shovel or broom or other pole supported implement, each without motors. This pole component should have area(s), referred to herein as handles, wherein a two armed user could hold the implement and work it without using the other one arm features. The elongated pole has an upper section, block 5 and a lower section, block 13. This division of upper and lower is arbitrary, so as to show relative positions of the other components on the pole. The pole upper section has a handle portion for use by a two armed person, and the handle may merely be an area of the pole onto which a two armed user holds the implement for use, such as usual raking or paint rolling, etc. Alternatively, this handle may be an area that has wrapping or a loop of hand fitting or other handle feature. Thus, the handle herein must be distinguished from the hand grip, block 9, which is on the pole but intended to aid one arm users. The upper pole section also has a forearm receptacle, block 11, described in more detail in FIG. 1B and other Figures below. The pole lower section, block 13, includes a work function tool, block 15, and at least one push plate, block 17. The work function tool is a generic expression for a tool head component, such as on an axe, snow shovel, paint roller, pitch fork, raking fork, rake, digging shovel, gardening implements, such as a weed whacker or wed puller, mop, broom, squeegee, brush, pick, leaf blower, hedge trimmer, etc. In one preferred embodiment, the work function tool is a locking receiver with a plurality of interchangeable tool heads.

FIG. 1B is a schematic diagram of details of the forearm receptacle component, block 21, of the present invention one arm implement for a user with a disabled arm. The forearm receptacle may be permanently attached to the pole upper section, block 23, or it may be removably attached to the pole upper section, block 25, in which case it could be used to retrofit other pole-based implements. The hand grip for the one arm user, block 27, for the forearm receptacle permanently attached to the pole, may be built-in, i.e. formed as part of and forward of the forearm receptacle (see FIG. 10 below), or it may be separate. Likewise, the hand grip for the one arm user, block 29, for the forearm receptacle removably attached to the pole, may be built-in, i.e. formed as part of and forward of the forearm receptacle (see FIG. 10 below), or it may be separate. Optionally, but preferred, is an adjustment mechanism to permit a user to fit the forearm receptacle to his/her arm size (forearm thickness), block 31.

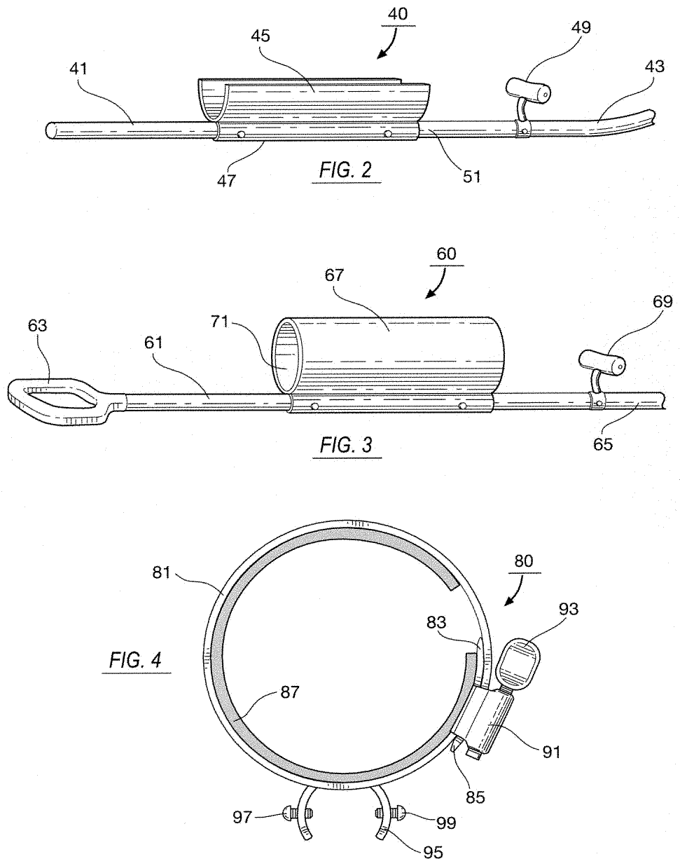

FIG. 2 shows a partial side view of an embodiment of a present invention one arm implement 40 for a user with a disabled arm, illustrating the pole 41, a handle area 51, and a grip 49 and arm receptacle 45 for one armed users (amputees, as well as permanently and temporarily arm disabled users). Receptacle 45 includes an attachment mechanism 47 being a tube and set screws, connected to the main frame of the receptacle 45 and attached to pole 41. Distal end 43 of pole 41 would include as least one tool, and a foot push plate near or on the tool, as described and/or shown in the prior and below Figures.

FIG. 3 shows a partial side view of another embodiment of a present invention one arm implement 60 for a user with a disabled arm, illustrating the pole 61, an upper handle 63 for two armed users, and grip 69 and arm receptacle 67 for one arm use. Distal end 65 of pole 61 would include as least one tool, as described and/or shown in the prior and below Figures.

FIG. 4 shows a front view of one embodiment of a present invention forearm receptacle component 80 having a semi-flexible main frame 81 with an extended internal comfort padding 87. Frame 81 has an internal end 83 wherein the padding 87 extends beyond end 83 as shown, to assure more padding for the user when frame 81 is tightened. External end 85 is partially wrapped around the opposite end 83 and includes an adjustment mechanism 91 for tightening or loosening the main frame 81 to fit the user. The main frame 81 has a series of slots near end 83 and a worm screw and housing that is fitted to the slots to permit rotation via key 93, in the manner of a hose clamp.

Also in FIG. 4, there is an attachment mechanism 95 connected (as welded or formed with metal or integrally cast or extruded with plastic) to frame 81, and has a plurality of attachment screws such as screws 95 and 97. This attachment mechanism yields slightly in either the opening or tightening direction, and can, thus, be used with different diameter poles.

All of the partial views above and below show details of some components and the other components not shown, such as at least one foot push plate and the tool at the distal end, are included by reference to full detail drawings shown herein.

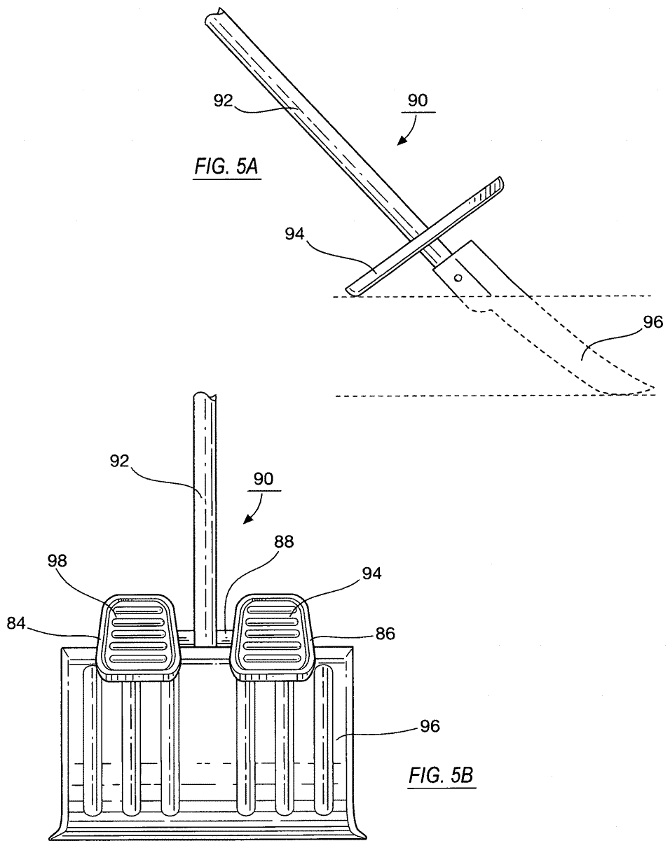

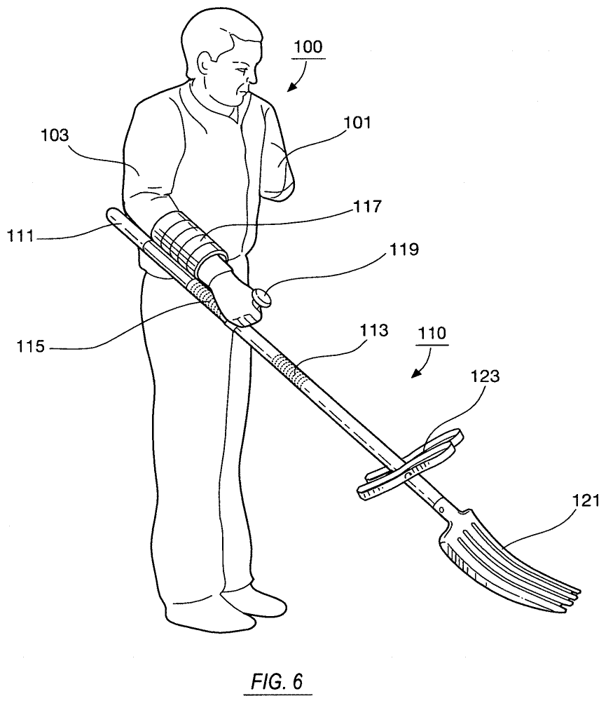

FIG. 5A shows a partial cut side view of a present invention one arm implement 90 for a user with a disabled arm and FIG. 5B shows a back view. These two Figures are now described collectively: These Figures show the foot push plate(s) 94 and 98 in conjunction with a snow shovel 96. These push plates are on both sides of pole 92 to permit a user to use his/her preferred foot (left or right). The push plates 94 and 98 are connected to the shovel tool and to the pole 92, and has a support yoke 88 connected to them and pole 92 for extra strength. At the upper section of the pole 90 are the handle, grip and forearm receptacle, not shown. FIG. 5B shows that the push plates 94 and 98 have raised rims 86 and 84 to prevent foot slippage when in use.

Foot push plates may alternatively be connected to only the pole or only the tool. In the case of shovels and other tools requiring significant pushing, the push plates are necessary for optimal effectiveness in accomplishing the tool-intended task. In the case of paint rollers and some other devices, the push plates are optionally used for putting additional pressure and guidance, such as when painting a garage or basement floor or a deck.

FIG. 6 shows a side view of and amputee 100 with a left arm 101 amputated, and the right arm 103 fully functional. He is using a present invention one arm implement 110, here, a rake or fork, such as may be used to muck hay and debris in a horse stall or to spread hay a on a newly seeded lawn. Implement 110 has a pole 111 with handle areas 113 and 115 for a two-armed user. Pole 111 also has a forearm receptacle 117 and grip 119 attached to or formed with pole 111, for amputee 100 (or someone, for example, with a broken arm in a sling). There are foot push plates 123 and distal end tool 121 with tines, as shown.

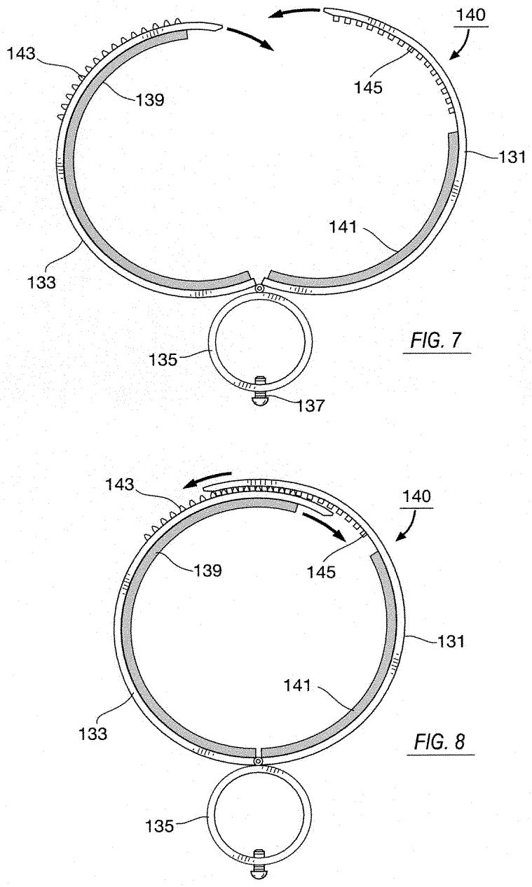

FIGS. 7 and 8 show a front, open and closed views, respectively, of another embodiment of a present invention forearm receptacle component 140 having extended internal padding section 141 and internal padding section 139, respectively on main frame arc wings 131 and 133. These wings 131 and 133 are molded or formed plastic or metal having slight flexibility and a unique adjustment mechanism due to the wings' biases and upward forearm pressure of a user. Wing 131 has a slightly smaller arc angle (radius) than wing 133. Thus, by pushing down slightly on wing 133 and sliding it under wing 131 (FIG. 8), there is an inherent locking mechanism caused by the teeth 143 and detents 145 interconnecting. The connection becomes stronger when a user uses the device due to the inherent upward force of the forearm against the upper area of the two wings 131 and 133. The user or an assistant may connect the two wings 131 and 133 within a broad range of positions for inside cross-section adjustment to accommodate different size forearms.

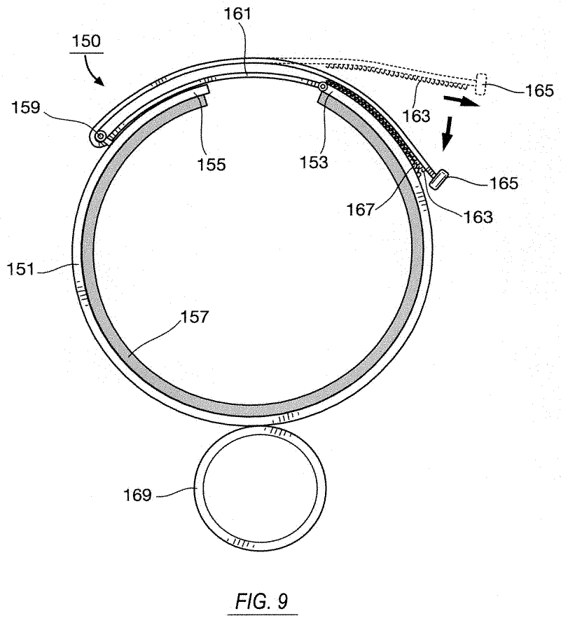

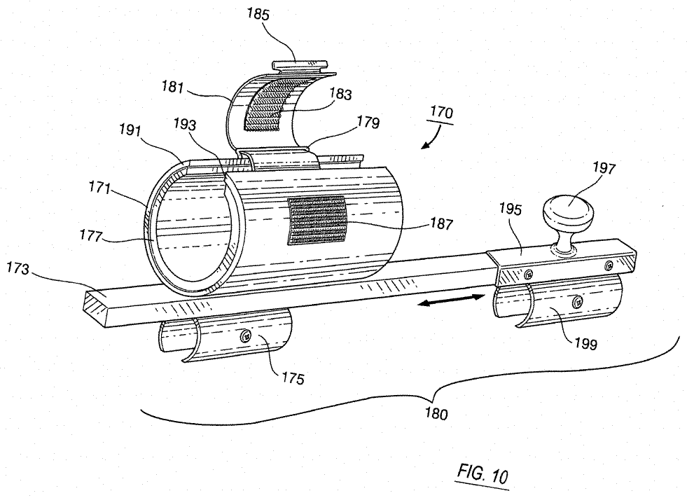

FIG. 9 shows show a front view of yet another embodiment of a present invention forearm receptacle component 150 having a main frame 151 with a first end 153 and a second end 155, and with extended internal padding 157. On the outside of main frame 151 near first end 153 is a Velcro strip, known as hook and loop fasteners. One of hooks and loops 167 is on the frame strip, and the other of hooks and loops 163 is on the inside of belt 161. Belt 161 is affixed to frame end 153 and passes through belt loop 159 and returns toward frame end 153, as shown. A one functional arm user may insert the good arm into the receptacle 150 and then the user or a third party can pull the belt 161 to the desired receptacle tightness and press to bind the hooks and loops. In the event that a disabled user is alone, teeth grip 165 may gripped between the teeth and pulled and pressed to lock into the desired tightness (desired diameter) of the frame 151. Likewise, disconnecting can be achieved by teeth grip 165 by bite and lift the grip 165 to release the hooks and loops connection. The belt 161 may be any functional belt, such as fabric, plastic, leather or fiberglass webbing. The frame 151 is semi flexible plastic or metal. There is an attachment ring 169 for fitting to a pole. Other components not shown as shown and described above and below.

FIG. 10 shows show an oblique side view of yet another embodiment of a present invention forearm receptacle and grip component 180 wherein the grip and the receptacle are shown s an integral component. Receptacle 170 has a main frame 171 with a first end 191 and a second end 193, and with internal padding 177. On the outside of main frame 171 is a hook and loop fastener. One of hooks and loops 187 is on the frame, and the other of hooks and loops 183 is on the inside of belt 181. Belt 181 is affixed to frame end 193 and passes through belt loop 179 and returns toward frame end 193, as shown. A one functional arm user may insert the good arm into the receptacle 170 and then the user or a third party can pull the belt 181 to the desired receptacle tightness and press to bind the hooks and loops. In the event that a disabled user is alone, teeth grip 165 may gripped between the teeth and pulled and pressed to lock into the desired tightness (desired diameter) of the frame 151. Likewise, disconnecting or loosening can be achieved by teeth grip 185 by bite and lift the grip 185 to release the hooks and loops connection. The belt 181 may be any functional belt, such as fabric, plastic, leather or fiberglass webbing. The frame 171 is semi flexible plastic or metal and the padding may be fabric, filled fabric, foam or combinations thereof.

Also shown in FIG. 10 is receptacle base 173 and attachment bracket 175 for attachment to a pole. Interconnected with receptacle base 173 is grip base 195, which is telescopically connected to base 173 to permit a user to adjust the distance between the receptacle 170 and the grip 197. Grip base 195 also has a pole attachment bracket 199 for fitting to a pole. Other components not shown as shown and described above and below. It should be noted that in this preferred embodiment, both the receptacle and the distance between the receptacle and the grip are both adjustable for maximum comfort and efficiencies.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims. For example, the actual shape or the length of the forearm receptacle may be any of numerous possibilities as long as its functionality as described, ergonomically acceptable, and it does not adversely affecting the functionality of other components.

What is claimed is:

1. A one-arm implement for a user with a disabled arm, which comprises:
a) an implement for performing a work function, said implement having an elongated pole with an upper section and a lower section, said upper section including a handle portion, and said lower section having a distal end, and having a work function tool located at said distal end;
determining the angle of the meridian axis of astigmatism of said eye relative to a reference axis;
b) a forearm receptacle located on said upper section of said pole;
c) a hand grip located on said upper section of said pole and positioned downwardly from said forearm receptacle;
d) at least one foot push plate located on at least one of: said lower section of said pole; and said work function tool.

2. The one-arm implement for a user with a disabled arm of claim 1, wherein said at least one foot push plate is located on said lower section of said pole.

3. The one-arm implement for a user with a disabled arm of claim 1, wherein said at least one foot push plate is located on said work function tool.

4. The one-arm implement for a user with a disabled arm of claim 1, wherein said at least one foot push plate is located on both said lower section of said pole and said work function tool.

5. The one-arm implement for a user with a disabled arm of claim 1, wherein said at least one foot push plate is at least partially located rearward of said on said lower section of said work function tool and forms an angle no greater than 60 degrees when measured at the at least partially located rearward section and up.

6. The one-arm implement for a user with a disabled arm of claim 2, wherein said at least one foot push plate is at least partially located rearward of said on said lower section of said work function tool and forms an angle no greater than 60 degrees when measured at the at least partially located rearward section and up.

7. The one-arm implement for a user with a disabled arm of claim 1, wherein said forearm receptacle and said grip are an integral unit connected to one another.

8. The one-arm implement for a user with a disabled arm of claim 7, wherein said forearm receptacle and said grip are an integral unit that are telescopically connected to one another for adjustment of distance between said forearm receptacle and said grip.

9. The one-arm implement for a user with a disabled arm of claim 1, wherein said work function tool is selected from the group consisting of a snow shovel, a digging shovel, a rake, a fork, a broom and a mop.

10. The one-arm implement for a user with a disabled arm of claim 1, wherein said work function tool is a shovel.

11. A one-arm implement for a user with a disabled arm, which comprises:
a) an implement for performing a work function, said implement having an elongated pole with an upper section and a lower section, said upper section including a handle portion, and said lower section having a distal end, and having a work function tool located at said distal end;
b) a forearm receptacle located on said upper section of said pole, said forearm receptacle including at least one opening-adjustment mechanism adapted to provide a user with an adjustable fitting to accommodate different thickness forearms;
c) a hand grip located on said upper section of said pole and positioned downwardly from said forearm receptacle;
d) at least one foot push plate located on at least one of: said lower section of said pole; and said work function tool.

12. The one-arm implement for a user with a disabled arm of claim 11, wherein said at least one foot push plate is located on said lower section of said pole.

13. The one-arm implement for a user with a disabled arm of claim 11, wherein said at least one foot push plate is located on said work function tool.

14. The one-arm implement for a user with a disabled arm of claim 11, wherein said at least one foot push plate is located on both said lower section of said pole and said work function tool.

15. The one-arm implement for a user with a disabled arm of claim 11, wherein said at least one foot push plate is at least partially located rearward of said on said lower section of said work function tool and forms an angle no greater than 60 degrees when measured at the at least partially located rearward section and up.

16. The one-arm implement for a user with a disabled arm of claim 12, wherein said at least one foot push plate is at least partially located rearward of said on said lower section of said work function tool and forms an angle no greater than 60 degrees when measured at the at least partially located rearward section and up.

17. The one-arm implement for a user with a disabled arm of claim 11, wherein said forearm receptacle and said grip are an integral unit connected to one another.

18. The one-arm implement for a user with a disabled arm of claim 17, wherein said forearm receptacle and said grip are an integral unit that are telescopically connected to one another for adjustment of distance between said forearm receptacle and said grip.

19. The one-arm implement for a user with a disabled arm of claim 11, wherein said work function tool is selected from the group consisting of a snow shovel, a digging shovel, a rake, a fork, a broom and a mop.

20. The one-arm implement for a user with a disabled arm of claim 11, wherein said work function tool is a shovel.
